(19) United States
(12) Patent Application Publication
(10) Pub. No.: US 2022/0347398 A1
(43) Pub. Date: Nov. 3, 2022

(54) Wireless Communication Enabled Drug Delivery Device and Method

(71) Applicant: AMGEN INC., Thousand Oaks, CA (US)

(72) Inventors: Sivakumar Paramanandam, Newbury Park, CA (US); Andrew Coles, Thousand Oaks, CA (US); Keng-Tong See, Camarillo, CA (US); Desheng Yin, Thousand Oaks, CA (US)

(21) Appl. No.: 17/621,135

(22) PCT Filed: Jun. 16, 2020
(86) PCT No.: PCT/US20/37839

§ 371 Date: Dec. 20, 2021

Related U.S. Application Data
(60) Provisional application No. 62/864,014, filed on Jun. 20, 2019.

(51) Int. Cl. A61M 5/50

(52) U.S. Cl. CPC A61M 5/5086; A61M 2205/8212; A61M 2205/3569

(57) ABSTRACT

This disclosure describes a drug delivery device with communication functionality for purposes of transferring information to a user device, such as a smartphone, while maintaining power-efficient operation. The drug delivery device comprises a controller configured to, while operating in the active mode, use one or more sensors to detect that the injection mechanism has performed an injection. The controller is also configured to generate in memory a data entry indicative of the injection and/or a state of the drug delivery device, and switch into the low-power mode subsequent to or contemporaneous with detecting that the injection mechanism has performed the injection. The drug delivery device also comprises a wireless communication module powered by the power source and configured to establish a wireless connection with and transfer a message to a user device while the controller is operating in the low-power mode.

### CROSS-REFERENCE TO RELATED APPLICATIONS

Priority is claimed to U.S. Provisional Patent Application No. 62/864,014, filed Jun. 20, 2019, the entire disclosure of which is hereby incorporated herein by reference.

### FIELD OF DISCLOSURE

This disclosure generally relates to drug delivery devices including those for performing subcutaneous injections, and, more particularly, to drug delivery devices having wireless communication capabilities.

### BACKGROUND

Drugs can be administered through the use of drug delivery devices, such as autoinjectors, on-body injectors, among others. These devices may replace traditional delivery devices such as a conventional syringe. Autoinjectors and on-body injectors may be used to automate the injection process, thereby simplifying the process for patients and making self-administration an option in certain cases. Certain automated drug delivery devices include sensors and other electronics for monitoring use of the device. Information collected by such sensors can be wirelessly communicated to an external device such as a smartphone so that this information can be displayed to a user, stored in a memory, or made available to a healthcare provider such as a physician. Establishing a wireless connection between the drug delivery device and the external device can take time, particularly if the devices are not initially within range of each other. The time spent by a drug delivery device searching for the external device can place significant power demands on a battery included in the drug delivery device. Increasing the size of the battery to meet this demand, however, is not desirable because this will change the form factor or shape of the drug delivery device, which in turn will affect handling of the device by a patient performing an injection. Moreover, a larger battery adds costs to the device and this increases the costs of medical care.

As set forth below in more detail, the present disclosure sets forth systems and methods for wirelessly communicating information with a drug delivery device embodying advantageous alternatives to existing systems and methods, and that may address one or more of the challenges or needs mentioned above, as well as provide other benefits and advantages.

### SUMMARY

This disclosure describes adding communication functionality to a drug delivery device for purposes of transferring information to a user device (e.g., a mobile computing device such as a smartphone, a personal computer, a server, etc.) while maintaining power-efficient operation. In one aspect, the drug delivery device comprises: a reservoir adapted to contain a drug, an injection mechanism coupled with the reservoir to deliver a drug from the reservoir, a power source, one or more sensors, a memory, a controller powered by the power source and having an active mode and a low-power mode. The controller is configured to, while operating in the active mode, use the one or more sensors to detect that the injection mechanism has performed an injection. The controller is also configured to generate in the memory a data entry indicative of the injection and/or a state of the drug delivery device, and switch into the low-power mode subsequent to or contemporaneous with detecting that the injection mechanism has performed the injection. The drug delivery device also comprises a wireless communication module powered by the power source and configured to establish a wireless connection with a user device while the controller is operating in the low-power mode, and transmit a message indicative of the injection and/or the state of the drug delivery device to the user device.

In another aspect, a method of operating a drug delivery device comprises detecting, by a controller operating in an active mode and communicatively coupled to one or more sensors, that an injection has been performed with the drug delivery device. The method also comprises storing in a memory a data entry indicative of the injection and/or a state of the drug delivery device, and switching the controller into a low-power mode subsequent to or contemporaneous with detecting that the injection has been performed. The method comprises establishing, while the controller is operating in the low-power mode, a wireless connection with a user device via a wireless communication module included in the drug delivery device. The method also comprises transmitting, by the wireless communication module and while the controller is operating in the low-power mode, a message indicative of the injection and/or the state of the drug delivery device to the user device.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 illustrates a system comprising a power-efficient drug delivery device configured to wirelessly communicate with a user mobile device.

FIG. 2 illustrates an exemplary interaction between a controller and a wireless communication module.

FIG. 3 is an exemplary state diagram depicting four power states of a drug delivery device.

FIG. 4 is a flow diagram of an exemplary method of power-efficient operation of a drug delivery device.

### DETAILED DESCRIPTION

The present disclosure relates to operating a drug delivery device with wireless communication capabilities in a power-efficient manner. The implementations described herein provide energy savings by efficiently managing the power consumption of a controller and/or a wireless communication module included in the drug delivery device. One or both of these components may be switched into and kept in a low power mode when the respective component is not contributing to or necessary for the current use or operation of the drug delivery device. Establishing a wireless connection with a user device may take substantial time due to unavailability or lack of proximity of the user device. Thus, the controller may stay in the low-power mode while the wireless communication module attempts to establish a wireless connection. Also, while the wireless communication module is attempting to establish the connection, the controller may, independently of the wireless communication module, switch between active and low-power modes when a user employs the drug delivery device for additional injections. A variety of triggering events corresponding to automatic tasks performed by the drug delivery device and to user actions may cause the switching between power modes and, consequently, enable power-efficient operation. So configured, the drug delivery device according to the present disclosure has an increased battery life.

Each of the foregoing components of the drug delivery device and methods of operating the drug delivery device will now be described in more detail.

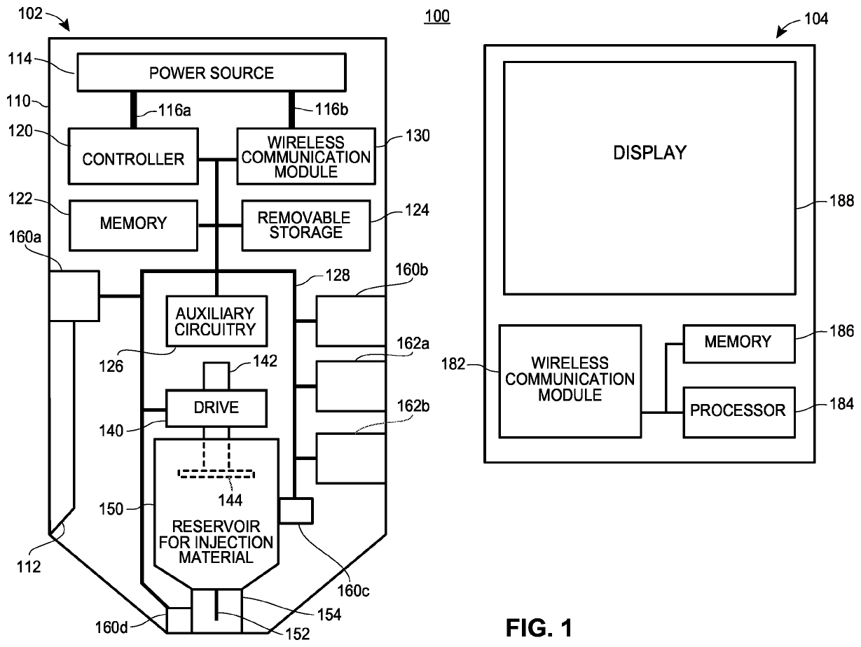

FIG. 1 illustrates a system 100 including a power-efficient drug delivery device 102 configured to wirelessly communicate with a user mobile device 104 (also referred to herein as a “mobile device” or “user device”). According to some embodiments, the drug delivery device 102 may be an autoinjector or other hand-held device configured to automatically or semi-automatically deliver a dose of injectable material (e.g., a medicine, a drug, a vaccine, or another therapeutic substance) to a user (e.g., a patient) via a subcutaneous injection. By virtue of its automation, the drug delivery device 102 may be easier and/or more convenient for a patient to use as compared to a manual injection device such as a conventional syringe. After coming in contact with skin at an injection site and receiving an input from a user, the drug delivery device 102 may automatically puncture the skin and subcutaneously deliver the injectable material. The drug delivery device 102 may thus help overcome physical and/or psychological difficulties associated with injections, improve the experience of a user or a patient, and/or increase compliance with a prescribed injection regimen. Additionally, the drug delivery device 102 may enable automatic record keeping of injections and, consequently, enhance the quality of medical care. To that end, the drug delivery device 102 may include electronic components that record information associated with injections and that transfer the recorded information to the user device 104, making the information available, for example, to a patient or a healthcare provider via a software application running on the user device 104. As discussed in more detail below, power-efficient operation of the drug delivery device 102 may reduce the need to recharge or replace a battery of the drug delivery device 102, reducing cost and environmental impact of operation, as well as further enhancing user experience. Furthermore, in some embodiments, the drug delivery device 102 may be reusable in the sense that it may be used to perform multiple injections. However, single-use or disposable embodiments of the drug delivery device 102 are also envisioned.

As seen in FIG. 1, the drug delivery device 102 may include a housing 110, within or on which are disposed a variety of components, assemblies, and/or structures for delivering injections, as well as for recording and communicating data associated with the injections and/or the state (e.g., an operational state) of the drug delivery device 102. The components disposed fixedly or removably within an interior space of the housing 110 may include a power source 114, power connections 116a, b, a controller 120, a memory 122, a removable storage device 124, auxiliary circuitry 126, a bus 128, a wireless communication module 130 (WCM), a mechanical drive 140, a plunger 142, a stopper 144, a reservoir 150 for injectable material, a needle 152, a cap 154, sensors 160a-d, and/or indicators 162a, b. The housing 110 may include flexible, articulating, and/or removable parts, such as a door 112, for example, for inserting and/or removing a cartridge that comprises the reservoir 150 of injectable material. The housing 110 may also include other doors to compartments with removable components, as well as openings or windows to provide, for example, visual, audio, and/or tactile access to some of the components, assemblies, and/or structures of the drug delivery device 102.

The power source 114 of the drug delivery device 102 may be one of the components removably attached to the housing 110 and accessible through a door (not shown), or may be fixedly attached to or disposed within a compartment of the housing 110. The power source 114 may be an electrical energy storage device, such as, for example a rechargeable lithium ion battery. In other implementations, the power source 114 includes one or more alkaline or other type of batteries, such as AA, AAA, 9V, coin cell or any other suitable type of batteries. Additionally or alternatively, the power source 114 may include one or more capacitors to increase power density of the power source 114. In some implementations, the power source 114 may be disposed outside of the housing 110 and be in mechanical and/or electrical connection with other components of the drug delivery device 102. The power source 114 may include two terminals that, in operation, maintain a substantially fixed voltage of 1.5, 3, 4.5, 6, 9, 12 V or any other suitable terminal voltage. The power source 114 may store an amount of charge, such as 100, 200, 500, 1000, 2000, 5000, 10000, 20000 mAh or any other suitable charge that can be delivered to one or more power-consuming loads as electrical current. In some implementations, the power source 114 is a rechargeable battery, configured to electrically connect to a charging circuit (not illustrated) that may be disposed at least partially outside of the drug delivery device 102.

The power source 114 may be in electrical connection with the controller 120 via the power connection 116a. Analogously, the power source 114 may be in electrical connection with the wireless communication module 130 via the power connection 116b. The power connections 116a, b may enable, respectively, the controller 120 and the wireless communication module 130 to draw power, charge, and/or current from the power source 114. Each of the power connections 116a, b may include switching and/or circuit protection devices that may be configured to limit or stop current drawn, respectively, by the controller 120 and/or the wireless communication module 130. The power connections 116a, b may also include components for regulating voltage (e.g., Zener diodes, transistor-based voltage regulators, etc.). Via additional power connections (not shown), the power source 114 may be in electrical connection with and supply power to a variety of other loads, including, for example, the memory 122, the removable storage device 124, the auxiliary circuitry 126, the mechanical drive 140, the sensors 160a-d, and/or the indicators 162a, b.

The controller 120 may include one or more processors, such as a microprocessor (μP), a digital signal processor (DSP), a central processing unit (CPU), a graphical processing unit (GPU), a field-programmable gate array (FPGA), an application-specific integrated circuit (ASIC), and/or any other suitable electronic processing components. Additionally or alternatively, the controller 120 may include a microcontroller (μC). The controller 120 may be in communicative connection with the memory 122. In some implementations, the memory 122 may be included in, integrated into, and/or be a part of the controller 122. In some of these implementations, the memory 122 included in the controller 120 may be disposed on the same chip or IC as a processor, in a system on a chip (SoC) configuration. In other implementations, the memory 122 may be disposed in a separate package from the one or more processors of the controller 120. The memory 122 in the controller 120 or in communicative connection with the controller 120 may include one or more electronic memory components, such as one or more registers, random access memory (RAM), read-only memory (ROM), electrically erasable programmable read-only memory (EEPROM), and/or flash memory. Additionally or alternatively, the controller 120 may be in communicative connection with the removable storage device 124, such, as a flash drive, an SD (secure digital) card, and/or a microSD card. The controller 120 may be configured to read and/or write information from and/or to the memory 122 or to internal memory. Analogously, the controller 120 may be configured to read and/or write information from and/or to the removable storage device 124. The controller 120 may be packaged and mounted on a circuit board and may interact with other components of the drug delivery device 102 using a plurality of connectors or pins.

According to those embodiments wherein the controller 120 is defined by a microprocessor or the like, the configuration of the controller 120 may correspond to the programming of the controller.

The wireless communication module 130 may include a wireless chip set. Whether implemented using a wireless chipset or not, the wireless communication module 130 may include one or more processors, such as a microprocessor, a DSP, a CPU, a GPU, an FPGA, an ASIC, and/or any other suitable electronic processing components. The wireless communication module 130 may include, in communicative connection with at least one processing component, one or more memory components, such as one or more registers, RAM, ROM, EEPROM, and/or on-board flash memory. The wireless communication module 130 may include one or more communication components including at least one transmitter and at least one receiver. The transmitter and receiver may be parts of a single transceiver unit. The transmitter and receiver may be configured to communicate at radio frequencies (RF) from 50 kHz to 100 GHz or using optical frequencies from infrared to ultraviolet, for example. The RF transmitters and receivers may comprise oscillators, amplifiers, filters, and/or antennas, while optical transmitters and receivers may comprise light-emitting diodes, lasers, photo-detectors, optical amplifiers, fibers, and/or lenses. The wireless communication module 130 may be configured to communicate over an Industrial, Scientific, and Medical (ISM) frequency band. In some implementations, the wireless communication module 130 is a WiFi, a near field communication (NFC), a Bluetooth, and/or Bluetooth low energy (BLE) module. The wireless communication module 130 may be packaged and mounted on a circuit board and may interact with other components of the drug delivery device 102 using a plurality of connectors and/or pins.

Power-efficient operation of the drug delivery device 102 may prolong the life of the power source 114, and/or reduce the frequency at which the power source 114 must be recharged, by reducing power consumption of some or all of the loads, such as the controller 120 and the wireless communication module 130. The controller 120 may be configured to operate in a plurality of modes with different power consumptions or current draws, including at least an active mode and a low-power mode. While operating in the active mode, the controller 120 and optionally other electronic components included in the drug delivery device 102 may consume or draw more power from the power source 114 than when operating in the low-power mode. In the active mode, the controller 120 may control operation of the drug delivery device 102, as described below, and/or may have access to all or most of the computing resources within the controller 120. Even when the controller 120 is idle, the ready access to the computing resources in the active mode may result in substantial power consumption. In contrast, in the low-power mode, the controller 120 may have only limited access to the computing resources of the controller 120 to save power. One example of the low-power mode of the controller 120 may be a case in which power to the controller 120 is cut off, for example, by a switch external to the controller 120. Another example may be a so-called “sleep” mode of the controller 120. In some embodiments, the controller 120 may draw less power from the power source 114 of the drug delivery device 102 in the low-power mode than in active mode. To transition from the low-power mode to the active mode, the controller 120 may require a trigger corresponding to an external action, such as, for example, switching on the power or sending a “wake-up” signal to the controller 120.

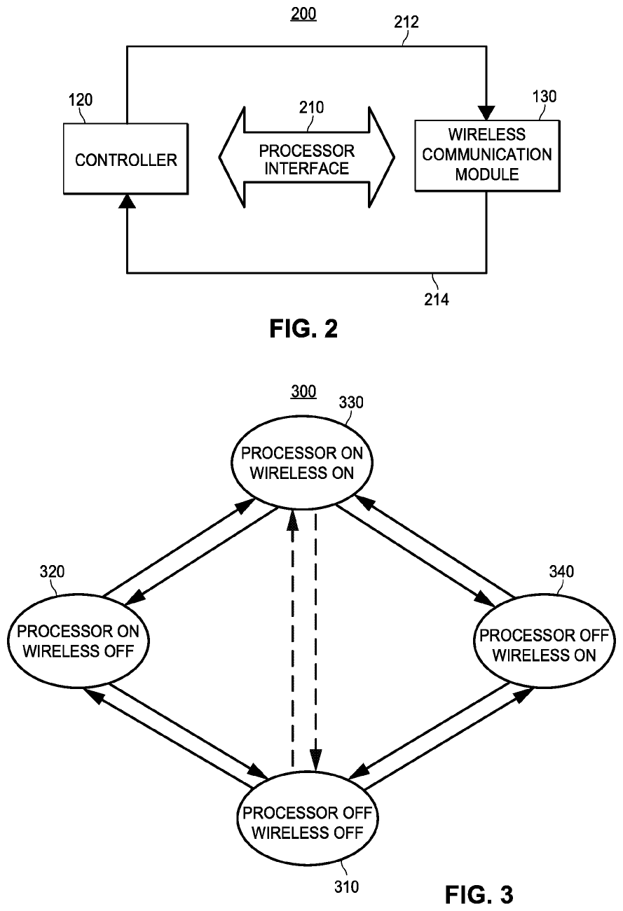

Analogously to the controller 120, the wireless communication module 130 may be configured to operate in a plurality of modes with different power consumptions or current draws, including active modes and low-power modes. In addition to enabling access to the computing resources within the wireless communication module 130, an active mode of the wireless communication module 130 may also enable wireless communication resources that substantially contribute to power consumption. In contrast, a low-power mode of the wireless communication module 130 may refer to an operating mode in which access to the computing resources and/or the wireless communication resources is limited to save power. As with the controller 120, the wireless communication module 130 may require a trigger corresponding to an external action to switch-on power or to wake up from a sleep mode and to transition into the active mode. More details about the controller 120 and the wireless communication module 130, as well as actions causing operating mode transitions, are discussed below in the context of FIG. 2, which illustrates possible interactions between the controller 120 and the wireless communication module 130.

Returning to FIG. 1, the controller 120 and the wireless communication module 130 may be in communicative connection with each other via a communication bus 128. The communication bus 128 may also communicatively connect other components including, for example, the memory 122, the removable storage device 124, the auxiliary circuitry 126, the mechanical drive 140, the sensors 160a-d, and/or the indicators 162a, b to the controller 120 and/or to the wireless communication module 130. The communicative interconnections among the components may be implemented using one or more circuit board traces, wires, and/or other electrical, optoelectronic, and/or optical connections. The communicative interconnections may be configured to carry signals that conform to any one or more of a variety of protocols, such as I2C, SPI, and/or other logic to enable cooperation of the various components, assemblies, and/or structures of the drug delivery device 102 in performing the functions of the drug delivery device 102, as described below.

The controller 120, while operating in an active mode, may read instructions from the memory 122 and execute the instructions to operate the drug delivery device 102. For example, the controller 120 may detect that the conditions are met for initiating and delivering an injection. Meeting the conditions for delivering the injection may include detecting that a user activated a finger sensor 160b, for example, by pressing, pushing, and/or covering the finger sensor 160b. The finger sensor 160b may include a button, a capacitive touch sensor, a light sensor, or any other suitable sensor that may detect a touch or close proximity of a finger, a palm, or another suitable object. Additionally or alternatively, meeting the conditions for delivering the injection may include detecting, using a skin sensor 160d, that the drug delivery device 102 is in contact with skin. The skin sensor may include a capacitance sensor, a resistance sensor, an inductance sensor, a pressure sensor, a light sensor, and/or any other suitable sensor configured to detect that the drug delivery device 102 is in contact with skin at an injection site. Still additionally or alternatively, meeting the conditions for delivering the injection may include detecting, with one or more sensors (including, for example a door sensor 160a and a reservoir sensor 160c), that the reservoir 150 for injectable material is in proper position and contains the injectable material, and that the door 112 of the housing 110 is closed. The reservoir 150, in some implementations, is a part of a removable cartridge. Meeting additional or alternative conditions for delivering the injection may include determining, using the sensors 160a-d and/or additional sensors, that the drug delivery device 102 is in correct orientation with respect to the user and/or to the gravitational field. Meeting one or more other suitable conditions may also, or instead, be required before the controller 120 causes the drug delivery device 102 to proceed with the injection.

After the controller 120 detects, while operating in the active mode and using sensors (e.g., the sensors 160a-d), that the conditions for delivering the injection are met, the controller 120 may engage or activate an injection mechanism to deliver the injection subcutaneously at the injection site on the skin of the user. According to some embodiments, the injection mechanism may correspond to one or more of the drive 140, the plunger 142, or other components included in the drug delivery device 102. The drive 140 may include an electric motor which, in operation, may draw current from the power source 114. To deliver the injectable material to the patient, the injection mechanism may be configured to provide the motive force for inserting the needle 152 into the patient and/or expelling the injectable material from the reservoir 150 and out through the needle 152. The needle 152 may be in fluid communication with the reservoir 150 or operable to be connected in fluid communication with the reservoir 150 prior to or as a result of operation of the injection mechanism. The needle 152 may be moveable relative to the housing 110 between an initial or storage state, where a pointed end of the needle 152 is disposed within the housing 110, and a delivery state, where the pointed end of the needle 152 protrudes through an opening in the housing 110 beyond an exterior surface of the housing 110 for insertion into a patient. The injection mechanism may be configured to deliver the injection in two steps: puncturing the patient's skin with the needle 152 by moving the needle 152 from the storage state to the delivery state in order to form a fluidic path between the reservoir 150 and subcutaneous tissue at the injection site, followed by expressing the injectable material from the reservoir 150 into the patient's subcutaneous tissue. In alternative embodiments, the injection mechanism may not provide the motive force necessary for moving the needle 152 from the storage state to the delivery state and instead this action may be provided by the user manually pushing a retractable needle guard into the interior space of the housing 110 to expose the pointed end of the needle 152.

The needle 152 may be a generally tubular member with a sharpened tip for penetrating a patients skin and/or other tissue. The needle 152 may have a hollow interior that is in fluid communication with the reservoir 150 or configured to be moved into fluid communication with the reservoir 150 during operation of the drug delivery device 102. In some implementations, the needle 152 may be staked to the reservoir 150 such that needle 152 does not move relative to the reservoir 150. In certain such implementations, the reservoir 150 may be prefilled with a drug by a manufacturer such that the arrangement takes the form a prefilled syringe. Alternatively, the reservoir 150 may be filled at the point of care by the user or patient and/or the needle 152 may not be initially staked to the reservoir 150. Furthermore, in some implementations, the reservoir 150 may be included as part of a removable cartridge that may be placed (e.g., by a user), prior to the injection, within the housing 110 of the drug delivery device 102 by way of the door 112 in the housing 110. In some implementations, the injection mechanism may include a spring-loaded actuator configured to push the entire cartridge towards the skin of the user, causing the needle 152 to penetrate the skin. In some implementations, the drive 140 actuates the plunger 142 to push the cartridge and/or cause the needle 152 to penetrate the skin. The cartridge or the housing of the drug delivery device 102 may contain the cap 154, which may cover the needle 152 of the cartridge prior to the injection. The cap 154 may need to be removed (e.g., by the user) prior to the injection, and meeting the conditions for delivering the injection may include the controller 120 detecting the absence of the cap 154 using a sensor not shown in FIG. 1.

The drive 140 may be configured to actuate the plunger 142 that may be in mechanical connection with a stopper 144 disposed within or forming a movable wall of the reservoir 150. In some applications, the stopper 144 may be a component of the cartridge that includes the reservoir 150, and the plunger 142, upon being actuated by the drive 140, may come into mechanical contact with the stopper 144. The actuated plunger 142 may move the stopper 144 towards the needle 152, forcing the injectable material out of the reservoir 150 and into the tissue through the needle 152. Upon forcing a required amount of the injectable material out of the reservoir 150 (which, in some implementations and/or application, includes substantially emptying the reservoir 150), the injection mechanism may retract the needle 152 from the skin of the user and back into the housing. The controller 120 may detect, while still operating in an active mode, and by using one or more sensors (e.g., sensors 160a-d), that the injection mechanism completed the injection. The controller 120 may use a visual indicator 162a and/or an audio indicator 162b to indicate to the user that the drug delivery device 102 completed delivering the injection. The visual indicator 162a may include a light emitting diode (LED), a liquid crystal display (LCD), or any other suitable visual indicator device. The audio indicator 162b may include a speaker, a buzzer, or any other suitable audio indicator device. After receiving an indication of a completed injection, the user may move the drug delivery device 102 away from the injection site, breaking the contact between the drug delivery device 102 and the skin. The controller 120 may detect the broken contact with skin using the skin sensor 160d and, in response to detecting the broken skin contact and after a pre-determined pause, turn off the indication of the completed injection.

The controller 120, still operating in the active mode, may create and record a digital data entry indicative of an injection in the memory 122 and/or on the removable storage device 124. The data entries may considerably enhance user experience with the drug delivery device 102 and user compliance with the injection regimen. Furthermore, the data entries may provide valuable clinical information to health service providers, drug manufacturers, and/or other stakeholders. Collectively, one or more data entries indicative of injections recorded by the controller 120 may be referred to as an injection log. The data entries in the injection log may include injection time/data, injection status, injection cartridge information, etc. Additionally or alternatively, the controller 120 may create one or more data entries indicative of status of the drug delivery device 102 in the injection log or in a different log (e.g., a maintenance log). The one or more data entries may include error codes generated during self-test routines, remaining charge of the power source 114, etc. The controller 120 may generate a data entry in the injection log subsequent and/or in response to detecting, using one or more sensors (e.g., sensors 160a-d), that the injection mechanism completed an injection and/or that the drug delivery device 102 lost contact with the injection site. In some implementations, the controller 120 detects using the sensors 160a-d, a failed injection attempt and generates a data entry in the injection log in response to the failed injection attempt. After generating the data entry, and also subsequent and/or in response to detecting that the injection mechanism completed an injection, the controller 120 may switch into a low-power mode. In some implementations, before switching into the low-power mode, the controller 120 communicates with the wireless communication module 130, using, for example, a processor interface. The controller 120 may, for example, transfer at least some data indicative of a completed or a failed injection and/or of a state of the drug delivery device 102 to the wireless communication module 130. Additionally or alternatively, the controller 120 may cause the wireless communication module 130 to switch into the active mode from the low-power mode, and/or cause the wireless communication module 130 to communicate with the user device 104, as described below.

To make the information in the injection log or the drug delivery device state data, which may be stored in the memory 122 and/or the removable storage device 124, more accessible and useful, the drug delivery device 102 may be configured to transfer at least a portion of the information or the data to the user device 104. The drug delivery device 102 may use the wireless communication module 130 to establish a wireless connection with the user device 104 and to transmit one or more messages, containing at least a portion of the injection log information, via that wireless connection. Additionally or alternatively, the wireless communication module 130 may obtain and transmit unrecorded information indicative of injections and/or the state of the drug delivery device 102. In some implementations and/or scenarios, the user device 104 receives at least some information that does not have a copy retained on the drug delivery device 102.

For example, the drug delivery device 102 may use the wireless communication module 130 to transmit injection status or drug delivery device state data without retaining the record of the transmissions or the data included in the transmissions at the drug delivery device 102. The injection status or the drug delivery device state data may include, for example, data indicative of motor or plunger position, data indicative of fullness of the reservoir 150, etc. In some implementations, the drug delivery device 102 may stream, using the wireless communication module 130, the injection status or the drug delivery device state data at regular time intervals (e.g., every 1, 10, 100, 1000 ms). In other implementations, the drug delivery device 102 may send, using the wireless communication module 130, the injection status or the drug delivery device state data in response to a change detected by one or more of the sensors 160a-d or other sensors. The user device 104 (described below in more detail), upon receiving, from the drug delivery device 102 by way of the wireless communication module 130, the injection status or the drug delivery device state data may store the received information and/or generate outputs to a user. For example, the user device 104 may generate audio (e.g., beeps, voice commands, etc.), visual (e.g., light emitting diode, graphics and/or text rendered on a display, etc.), or haptic (e.g., vibration) signals to indicate to the user relevant information based on the data received from the drug delivery device 102. The signals generated by the user device may, for example, guide the user in performing the injection. The user may take actions (e.g., pause, continue, or correct user-implemented injection steps) that cause the drug delivery device 102 to change the state, triggering, in some implementations, new communications. In this manner, the wireless communication module 130 may facilitate interactive feedback between the drug delivery device 102 and the user during an injection or during other user-performed actions (e.g., replacing batteries, replacing a cartridge, setting time, etc.).

The wireless communication module 130 may be configured to accomplish, while operating in an active mode, the transfer of information to the user device 104 in a collection of steps that may be performed asynchronously or in sequence. The wireless communication module 130 may, for example, establish a wireless connection with the user device 104, obtain at least some data from a data entry indicative of an injection and/or a state of the drug delivery device 102, and transmit a message indicative of or comprising the obtained data to the user device 104. To establish the wireless connection with the user device 104, the wireless communication module 130 may initiate a sequence of one or more communication attempts. Each communication attempt may include one or more wireless transmissions containing, for example, some information identifying the drug delivery device 102 and an indication of an attempt to establish a connection with the user device 104. The communication attempts may be referred to herein as an “advertising” operation of the wireless communication module 130.

To begin advertising, the wireless communication module 130 may detect some triggering event, i.e. a communication trigger or, simply, “trigger,” and initiate the sequence of one or more communication attempts in response to detecting the communication trigger. In some implementations, the controller 120 sends a signal indicative of the communication trigger to the wireless communication module 130 via a UART (universal asynchronous receiver/transmitter) or another processor interface. The controller 120 may send the trigger at least in part in response to detecting that the injection mechanism completed an injection and/or detecting a specific user action. The user action for triggering communication may include opening and/or closing of the door 112, pressing a button, and/or activating the finger sensor 160b, for example. The wireless communication module 130 may detect the communication trigger based on a user action while the controller 120 is in the low-power mode. For example, auxiliary circuitry 126 may include digital logic that generates the communication trigger based on detecting the user action using the sensors 160a-d.

In some implementations and/or scenarios, the wireless communication module 130 need not receive any triggering signals from the user device 104, and simply transmits the message payload as a broadcast. The controller 120 and/or the wireless communication module 130 may encrypt the message prior to transmission using one or more encryption techniques, for example, to protect the privacy of the user. After broadcasting the message one or more times and/or repeatedly for a prescribed time interval, the wireless communication module 130 may switch into the low-power mode. After switching into the low-power mode, the wireless communication module 130 may power up, activate, and/or wake up in response to receiving a wireless activation trigger. The controller 120, for example, may send the wireless activation trigger to the wireless communication module 130 at least in part in response to detecting that the injection mechanism completed an injection and/or detecting a specific user action. The user action for triggering wireless activation may include opening and/or closing of the door 112, pressing a button, and/or activating the finger sensor 160b. Additionally or alternatively, the wireless communication module 130 may detect the wireless activation trigger based on a user action while the controller 120 is in the low-power mode. For example, auxiliary circuitry 126 may include digital logic that generates the wireless activation trigger based on detecting the user action using the sensors 160a-d. The wireless activation trigger may be the same as the communication trigger described above. Notably, the wireless communication module 130 may detect the wireless activation trigger, while operating in the low-power mode, and switch into the active mode in response to detecting the trigger.

The wireless communication module 130 may be configured to receive acknowledgements, confirmations, and/or other wireless signals transmitted by the user device 104. At least some steps in the collection of steps for establishing communication with the user device 104 and/or transmitting a message to the user device 104 may be in response to these signals transmitted by the user device 104. Thus, the discussion of the user device 104 presented below precedes the continued discussion of steps performed by the wireless communication module 130 of the drug delivery device 102.

The user device 104 may also comprise a wireless communication module 182 configured to communicate with the wireless communication module 130 of the drug delivery device 102 over a wireless link (e.g., a radio-frequency (RF), optical, acoustic link, etc.). The user device 104 may further comprise a processor 184, a memory 186, and a display 188. The display 188 may be a touch screen, configured to receive tactile input from the user, for example. The wireless communication module 182 of the user device may be in communicative connection with the processor 184 and/or the memory 186. The processor 184 of the user device 104 may execute instructions stored in the memory 186 of the user device 104. For example, the instructions may comprise a software application, and cause the processor 184 to retrieve and process information transferred to the user device 104 from the drug delivery device 102. Upon retrieving the information (e.g., data indicative of an injection, data indicative of a state or status of the drug delivery device 102, etc.) from the drug delivery device 102, the processor 184 may store at least some of the information (e.g., a record of an injection) in the memory 186 and/or another digital storage module (not shown) of the user device 104. The processor 184 may then cause the display 188 of the user device 104 to display to a user, based on the information transferred from the drug delivery device 102, an informational prompt. The informational prompt may be an indication of the state of the drug delivery device 102, a record of one or more injections, etc. The user device 104 may process the information received from the drug delivery device 102 to enhance the information available to the user via a user interface rendered on the display 188. The displayed information may enable a user to see the time of the last injection, the success record and/or compliance record for previous injections, and/or other useful information.

In response to detecting a wireless transmission from the wireless communication module 130 of the drug delivery device 102, the wireless communication module 182 of the user device 104 may transmit a response (e.g., a response message, or another suitable electronic signal having a specific format). In some implementations, the wireless communication module 182 of the user device 104 transmits the response at least in part based on the information identifying the drug delivery device 102 contained in the transmission from the wireless communication module 130 of the drug delivery device 102. In other implementations, the user device 104 may transmit an identifying signal that is not in response to any transmissions from the drug delivery device 102. For example, the transmission from the user device 104 may be in response to a user action associated with an application running on the user device 104.

In general, the transfer of information indicative of injections and/or state of the drug delivery device 102 may be contingent on authentication of the user device 104 by the drug delivery device 102 and/or authentication of the drug delivery device 102 by the user device 104. User actions may be required to establish a first or original authentication, as a prerequisite to creating a pairing, between the user device 104 and the drug delivery device 102. If the drug delivery device 102 and the user device 104 are paired, establishing communication and the transfer of information between the drug delivery device 102 and the user device 104 may proceed automatically, i.e., without additional user actions. To explain further, the drug delivery device 102, using the wireless communication module 130, may establish a connection with a paired user device 104 that is in range and available, and may transfer information to the paired user device 104 in response to certain triggering events other than user actions (e.g., in response to a signal that is broadcast by user device 104).

The communication between the wireless communication module 130 and the user device 104 may be encrypted using one or more encryption methods. The encryption may use symmetric and/or public keys. The wireless communication module 130 and the user device 104 may use Wired Equivalency Privacy (WEP), Wi-Fi Protected Access (WPA), and/or WPA2 wireless security protocols. In some implementations, to authenticate the wireless communication module 130 and/or the user device 104, the wireless communication module 130 and/or the user device 104 connect to an authentication server. Additionally or alternatively, the wireless communication module 130 and/or the user device 104 may use password protection and/or biometric screening to authenticate a user.

As discussed above, the drug delivery device 102 may transfer information to the user device 104 via a wireless connection established between corresponding wireless communication modules 130, 188, based on certain triggering events. For example, the controller 120 of the drug delivery device 102 may be configured to detect that the injection mechanism completed an injection (e.g., by processing signals generated by one or more of the sensors 160a-d). Upon detecting the completed injection, the controller 120 may generate in the memory 122 a data entry indicative of the injection and trigger the wireless communication module 130 to transfer information associated with that data entry to the user device 104. In some implementations, the wireless communication module 130 may itself detect the completion of the injection using, for example, one or more of the sensors 160a-d and/or the auxiliary circuitry 126. Thus, the wireless communication module 130 may commence transferring information to the user device 104 upon the completion of the injection without receiving any signals (e.g., a trigger and/or any notification) from the controller 120.

Establishing a wireless connection necessary for transferring information from the drug delivery device 102 may require that there is a ready user device 104 in sufficient proximity to the drug delivery device 102 (i.e., in range). The readiness of the user device 104 may require that the user device 104 is powered on, paired to the drug delivery device 102, and/or running a suitable application. In some scenarios and/or implementations, the wireless communication module 130 may initiate a sequence of one or more communication attempts to establish the wireless connection with the user device 104. The wireless communication module 130 may receive an acknowledgement of the communication attempts from the user device 104. If there is no ready user device 104 in range (e.g., the wireless communication module 130 does not receive an acknowledgement from the user device 104), then the wireless communication module 130 may cease the communication attempts after a predetermined time period that may be referred to as a “timeout period.” After a timeout, i.e. after ceasing the communication attempts upon expiration of the timeout period without establishing a connection, the wireless communication module 130 may initiate, after another time period that may be referred to as a “communication pause,” another sequence of communication attempts. The wireless communication module 130 may continue the cycle of timeouts and renewed communication attempts for a duration of time that may be referred to as a “communication time window.” The wireless communication module 130 may determine that the communication time window expired, and, in response to determining that the communication time window expired, cease communication attempts until detecting another triggering event and switch into the low-power mode. The communication time window may have a duration of 1, 2, 4, 12, 24, 48, 72 hours or any other suitable time period. The timeout period may have a duration of 10 ms, 100 ms, 1 s, 10 s, 1 min, 10 min or any other suitable time period. The communication pause may have a duration of 10 s, 1 min, 10 min, 1 hr or any other suitable time period. In some implementations, the wireless communication module 130 determines that the communication time window expired by determining that the wireless connection with the user device 104 was not established after a time period greater than a threshold time period equal to the communication time window. Additionally or alternatively, the wireless communication module 130 may determine that the communication time window expired by determining that the wireless connection with the user device 104 was not established after a number of attempts greater than a threshold number of attempts.

The wireless communication module 130 may also cease communication attempts and/or switch into the low-power mode at least in part in response to determining that the wireless communication module 130 successfully established a wireless connection with the user device 104. The user device 104 may transmit an acknowledgement of an established wireless connection with the drug delivery device 102. The wireless communication module 130 may, in turn, receive, while operating in the active mode, the acknowledgement from the user device 104 and, at least in part in response to receiving the acknowledgement, switch into the low-power mode. Before switching into the low-power mode, and after receiving the acknowledgement, the wireless communication module 130 may transmit a message. The transmitted message may, for example, be indicative of at least some data obtained by the wireless communication module 130 from the controller 120, from memory 122, and/or from the removable storage device 124. The obtained data may contain at least some data from a data entry generated by the controller 120 and indicative of an injection and/or the state or status of the drug delivery device 102. Regardless of the content of the transmitted message, the user device 104 may transmit a confirmation in response to receiving the transmitted message. In response to receiving the confirmation, the wireless communication module 130 may switch into the low-power mode.

FIG. 2 illustrates an exemplary interaction 200 between the controller 120 and the wireless communication module 130 of the drug delivery device 102. In other embodiments, the controller 120 and the wireless communication module 130 of FIG. 2 are used in some other system or device. The interaction 200 may enable the controller 120 and the wireless communication module 130 to exchange information and/or to send trigger signals for switching between active and low-power modes.

As discussed above, the controller 120 and the wireless communication module 130 may each draw power from the power source 114. Thus, it may be advantageous to configure the drug delivery device 102 to substantially minimize power consumption by reducing the current draw of the controller 120 and/or the wireless communication module 130 when the corresponding component is not in use. Furthermore, it may be advantageous to configure the drug delivery device 102 to substantially minimize the use times of the controller 120 and/or the wireless communication module 130, while providing the needed functionality and a high-quality user experience. Thus, the controller 120 and/or the wireless communication module 130 may be configured to operate in different modes of operation with different power consumption profiles. The power consumptions of the controller 120 and/or the wireless communication module 130 are substantially proportional to corresponding current draws. In the discussion below, the exemplary current draws are, unless otherwise specified, current draws averaged over at least a significant fraction (e.g., greater than 5%) of the time spent in the corresponding operating mode before switching into another mode.

The controller 120 may be configured to operate in an active mode, for example, while controlling an injection mechanism, monitoring the sensors 160a-d, generating signals for the indicators 162a, b, writing to or reading from the memory 122 and/or the removable storage device 124, and/or sending signals to the wireless communication module 130. The current draw of the controller 120 operating in the active mode may be 0.05, 0.1, 0.2, 0.5, 1, 2, 5, 10 mA (milliamperes) or any other suitable current draw. The controller 120 may operate in a low-power mode, for example, while using a limited set of resources of the controller 120. The low-power mode of the controller 120 may be, for example, a stop mode, a standby mode, a sleep mode, a hibernation mode, or a shutdown mode. In some implementations, the controller 120 is configured to operate in one of a selection of low-power modes, depending on a scenario. The current draw of the controller 120 operating in one of one or more possible low-power modes may be 0.01, 0.02, 0.05, 0.1, 0.2, 0.5, 1, 2, 5, 10, 20 μA (microamperes) or any other suitable current draw. The current draw of the controller 120 operating in the active mode may be 2, 5, 10, 20, 50, 100, 200, 500, 1000, 2000, 5000, 10000 or any other suitable multiplicative factor higher than the current draw of the controller 120 in the low-power mode. For example, the controller 120 may draw more than 1 mA in the active mode and less than 10 μA in the low-power mode. In some implementations, a switch is configured to disconnect the controller 120 from the power source 114 reducing the current draw of the controller 120 operating in the resulting low-power mode substantially to zero.

The wireless communication module 130 also may be configured to operate in one of different modes with different corresponding current draws, and, consequently, different corresponding power consumption values. Operating in an active mode, the wireless communication module 130 may transmit and/or receive wireless signals. Additionally or alternatively, operating in an active mode, the wireless communication module 130 may, for example, prepare wireless transmissions, retrieve data for wireless transmissions from the memory 122 and/or external storage device 124, monitor the sensors 160a-d, and/or communicate with the controller 120. In the active mode, the wireless communication module 130 may draw a current of 0.1, 0.2, 0.5, 1, 2, 5, 10, 20, 50, 100, 200, 500 mA or any other suitable current while transmitting and/or receiving wireless signals. The wireless communication module 130 may be configured to send and/or receive wireless signals during only a fraction of the time spent operating in the active mode. Operating in the active mode while not transmitting and/or receiving, the wireless communication module 130 may draw 1, 2, 5, 10, 20, 50, 100, 200, 500 μA or any other suitable current. In a low-power mode, however, the wireless communication module 130 may draw 0.01, 0.02, 0.05, 0.1, 0.2, 0.5, 1, 2, 5 μA or any other suitably low current. The current draw of the wireless communication module 130 operating in the active mode may be 2, 5, 10, 10, 50, 100, 200, 500, 1000, 2000, 5000, 10000 or any other suitable multiplicative factor higher than the current draw of the wireless communication module 130 in the low-power mode. For example, the wireless communication module 130 may draw more than 5 mA in the active mode and less than 5 μA in the low-power mode. In some implementations, a switch is configured to disconnect the wireless communication module 130 from the power source 110, reducing the current draw of the wireless communication module 130 operating in the resulting low-power mode substantially to zero. The low-power mode of the wireless communication module 130 may be, for example, a standby mode, a stop mode, a sleep mode, a hibernation mode, or a shutdown mode. In the low-power mode, the wireless communication module neither transmits nor receives wireless signals.

The controller 120 and the wireless communication module 130 may be configured to communicate with each other via a processor interface 210. The processor interface 210 may use, for example, the bus 128 or another suitable physical connection. The processor interface 210 may include a parallel or a serial interface. In some implementations, the processor interface 210 includes a UART circuit in the controller 120 and/or the wireless communication module 130. In some implementations, the UART circuit is configured on general purpose input/output (GPIO) pins of the controller 120 and/or the wireless communication module 130 or, more specifically, on the GPIO pins of one or more of the processing components of the controller 120 and/or the wireless communication module 130. The processor interface 210 may employ protocols with flow control that include request to send (RTS) and clear to send (CTS) signals as well as transmit (Tx) and receive (Rx) signals.

The controller 120 may be configured to notify the wireless communication module 130 that the drug delivery device 102 completed an injection. In some scenarios or implementations, the controller 120 initiates communication with the wireless communication module 130, for example, by transmitting an RTS signal over a UART. The controller 120 may send a message informing of injection completion in response to receiving from the wireless communication module 130 a CTS signal over the UART. The message may include at least some data from a data entry indicative of the injection. Additionally or alternatively, the message sent by the controller 120 may include a communication trigger, and the wireless communication module 130 may initiate a sequence of attempts at connecting and/or communicating with the user device 104 in response to detecting the communication trigger. In some scenarios or implementations, the wireless communication module 130 may request from the controller 120 additional information, for example, indicative of one or more previous injections, the state or status of the drug delivery device 102, and/or past user actions. The wireless communication module 130 may initiate the request by transmitting an RTS signal over a UART, and, upon receiving a CTS signal from the controller 120, may transmit the full request message. After sending the requested information to the wireless communication module 130, the controller 120 may switch into the low-power mode.

In some implementations, the controller 120 and the wireless communication module 130 may send each other trigger signals via the processor interface 210 to switch the other component from active mode to low-power mode, and/or vice-versa. For example, the controller 120 may send the wireless communication module 130 an activation trigger signal via the processor interface 210. The wireless communication module 130, while operating in the low-power mode, may detect the activation trigger signal sent by the controller 120 via the processor interface 210, and, in response to detecting the activation trigger signal, switch into the active mode. Analogously, the wireless communication module 130 may send the controller 120 an activation trigger signal via the processor interface 210. The controller 120, while operating in the low-power mode, may detect the controller activation trigger signal sent by the wireless communication module 130 via the processor interface 210, and, in response, switch into the active mode. In some implementations, the trigger is an RTS signal sent by the controller 120 to the wireless communication module 130 or vice-versa.

In some implementations, the processor interface 210 is unavailable for communication when the controller 120 is in the low-power mode or when the wireless communication module 130 is in the low-power mode. The controller 120, by sending a signal via an activation line 212 distinct from the processor interface 210, may cause the wireless communication module 130 to switch into the active mode from the low-power mode. Analogously, the wireless communication module 130, by sending a signal via another activation line 214 distinct from the processor interface 210, may cause the controller 120 to switch into the active mode from the low-power mode. The activation line 212 is at times referred to herein as the “wireless communication module activation line.” The activation line 214 is at times referred to herein as the “controller activation line.” The activation lines 212, 214 may be parts of the bus 128, separate circuit board traces, or other communicative connections between the controller 120 and the wireless communication module 130. In some implementations, the controller 120 and/or the wireless communication module 130 may each be configured to detect trigger signals on multiple activation lines. A trigger detected on one of the multiple activation lines may correspond to one of multiple low-power modes, for example. Additionally or alternatively, at least some of the activation lines may convey signals sent by sources other than the controller 120 and/or the wireless communication module 130. For example, auxiliary circuitry 126 may send trigger signals based on user actions detected using the sensors 160a-d. Still additionally or alternatively, the activation lines 342, 344 may combine multiple trigger sources, e.g., the controller 120, the wireless communication module 130, and/or auxiliary circuitry 126, using digital logic.

In some implementations, triggers sent via the activation lines 342, 344 control, using switches, electrical connectivity between the power source 114 and the controller 120 and/or the wireless communication module 130. For example, the controller 120 or the auxiliary circuitry 126 may send a trigger to open and/or close a switch in the power connection 116a connecting the power source 114 to the wireless communication module 130. Analogously, the wireless communication module 130 or the auxiliary circuitry 126 may send a trigger to open and/or close a switch in the power connection 116b connecting the power source 114 to the controller 120 (or 120).

As discussed above, the controller 120 and/or the wireless communication module 130 may detect trigger signals received via the activation lines 212, 214, and switch into corresponding low-power modes in response to the detected triggers. In the low-power modes, the controller 120 and/or the wireless communication module 130 may monitor a limited set of connections or pins to detect activation triggers, may remove power from RAM and/or other peripherals, and/or may suspend execution of one or more processes. Additionally or alternatively, the wireless communication module 130 operating in the low-power mode may turn off amplifiers and/or other active RF and/or optical components in the transmitter and/or receiver of the wireless communication module 130. The configuration and the corresponding power consumption of a low-power mode may depend on which low-power mode (e.g., stand-by, sleep, hibernation, radio silence, power-off, etc.) is selected, for example by the trigger detected on the corresponding pin. Analogously, in some implementations, the configuration and the corresponding power consumption of an active mode may depend on which active mode is selected, for example by the trigger detected on the corresponding pin.

FIG. 3 is an exemplary state diagram 300 depicting four power states of the drug delivery device 102, corresponding to two power modes of the controller 120 and two power modes of the wireless communication module 130. There may be additional or alternative power states of the drug delivery device 102 when, for example, either the controller 120 or the wireless communication module 130 has more than two modes with different power consumptions. States 310-340 in FIG. 3 refer specifically to power states of the drug delivery device 102, and may be parts of state or status of the drug delivery device 102 recorded by the controller 120. The “both-off” state 310 of the drug delivery device 102 may represent the drug delivery device 102 with the controller 120 operating in the low-power mode of the controller 120 and the wireless communication module 130 operating in the low-power mode of the wireless communication module 130. The “controller-on” state 320 of the drug delivery device 102 may represent the drug delivery device 102 with the controller 120 operating in the active mode of the controller 120 and the wireless communication module 130 operating in the low-power mode of the wireless communication module 130. The “both-on” state 330 of the drug delivery device 102 may represent the drug delivery device 102 with the controller 120 operating in the active mode of the controller 120 and the wireless communication module 130 operating in the active mode of the wireless communication module 130. The “wireless-on” state 340 of the drug delivery device 102 may represent the drug delivery device 102 with the controller 120 operating in the low-power mode of the controller 120 and the wireless communication module 130 operating in the active mode of the wireless communication module 130. The drug delivery device 102 may indicate to a user, using the indicators 162a, b, the power state 310-340 of the drug delivery device 102 or a transition from one state to another.

The drug delivery device 102 may be in the both-off state 310 while in cold storage prior to initial use, between injections, or while charging the power source 110, for example. A user action, detected, for example, by one of the sensors 160a-d, may cause the drug delivery device 102 to transition to the controller-on state 320. In some implementations, the controller 120 switches into the active mode upon detecting that a user pushed a button (e.g., a power button, a cartridge eject button, etc.) or activated a different type of finger sensor (e.g., the finger sensor 160b). In another implementation, the controller 120 switches into the active mode upon detecting that the door 112 was opened and/or a cartridge with injectable material has been replaced. In yet another implementation, the controller 120 switches into the active mode upon detecting movement of the drug delivery device 102, using, for example, one or more accelerometers and/or other movement or vibration sensors. The drug delivery device 102 may be configured to activate the wireless communication module 130 substantially at the same time as activating the controller 120. Thus, from the both-off state 310, the drug delivery device 102 may transition into the controller-on state 320 or, in some implementations, directly into the both-on state 330. In some scenarios, as discussed below, the drug delivery device 102 also may transition into the wireless-on state 340 directly from the both-off state 310.

In some implementations, the transition into the controller-on state 320 or the both-on state 330 causes the controller 120 to run a diagnostic routine or a self-test. The diagnostic routine may verify operational readiness of the drug delivery device 102 by determining the remaining energy and/or charge in the power source 114 and/or by detecting error flags from the controller 120, the wireless communication module 130, and/or auxiliary circuitry 128. Additionally or alternatively, the diagnostic routine may verify that the sensors 160a-d are operational. Upon completing the diagnostic routine, the controller may generate a data entry indicative of the state of the drug delivery device and indicate to the user, using the indicators 162a, b whether the drug delivery device 102 is ready for normal use or requires troubleshooting. The normal use of the drug delivery device 102 may include delivering an injection, indicating the state or status of the drug delivery device 102 to the user, and/or sending information to the user device 104.

In some scenarios, with the drug delivery device 102 in the controller-on state 320 or the both-on state 330, the user may proceed with an injection. The user may, for example, load a cartridge, close the door 112, remove the cap 154, bring the drug delivery device 102 in contact with skin at an injection site, and press a button or engage the finger sensor 160b to cause the controller 120 to activate the injection mechanism which may include the drive 140 and the plunger 142. After at least some of the user actions, the controller 120 may detect, using the sensors 160a-d, that the actions were completed correctly and may notify the user, using the indicators 162a, b, that the user should proceed to the following step. For example, the controller 120 may detect, using the skin sensor 160d, that the drug delivery device 102 maintains injection site contact, and indicate to the user using the indicators 162a, b that the drug delivery device 102 is ready to inject. The controller 120 may be configured to make a data entry or, at least, to start a data entry in the memory 122 outside of the controller 120, and/or on the removable storage device 124 after any one of the user actions corresponding to the steps of the injection.

After activating the injection mechanism, the controller 120, still operation in the active mode, may monitor the sensors 160a-d to detect that the injection mechanism completed the injection. In some implementations, detecting that the injection mechanism completed the injection includes detecting that the skin sensor 160d lost contact with the skin and/or that the needle 152 is fully retracted. Additionally or alternatively, detecting that the injection mechanism completed the injection may include detecting, using the skin sensor 160d, that the drug delivery device 102 was in proximity to or in mechanical contact with the skin at the injection site for a predetermined time duration after the activation of the injection mechanism. The predetermined time duration may correspond to the time it takes the plunger 142 to traverse the distance necessary to express the injectable material from the reservoir 150. The predetermined time may depend on injection speed which may be pre-selected by the user. Additionally or alternatively, detecting that the injection mechanism completed the injection may include detecting, using the reservoir sensor 160c that the reservoir 150 has been substantially emptied of the injectable material. Still additionally or alternatively, detecting that the injection mechanism completed the injection may include detecting that the user has maintained contact with the finger sensor 160b or another button for a pre-determined time duration which may be different from the pre-determined time duration for maintaining skin contact. Furthermore, detecting that the injection mechanism completed the injection may include detecting whether the injection was completed successfully and, if not, determining a failure mode of the injection. Upon detecting that the injection completed, successfully or otherwise, the controller 120 may cause the door 112 to open, for example, to facilitate the removal of a used cartridge.

Subsequent and/or in response to detecting that the injection mechanism completed the injection, successfully or otherwise, the controller 120 may switch into the low-power mode of the controller 120, causing the transition out of the controller-on state 320 or the both-on state 330 into the both-off state 310 or the wireless-on state 340, respectively. Before switching into the low-power mode and after detecting the completion of the injection, the controller 120 may perform one or more tasks. For example, the controller 120 may generate in the memory 122 and/or in the removable storage device 124 a data entry indicative of the completed injection. In some implementations, the controller 120 adds to a previously generated data entry upon completing the injection. The data entry may include, for example, a timestamp indicative of when the injection was completed. Additionally or alternatively, the data entry may include timestamps for the various steps of the injection, information about the injectable material and/or the cartridge used in the injection, diagnostic information for the drug delivery device 102, and/or any other suitable information.

Before switching into the low-power mode, the controller 120 may generate one or more trigger signals for activating the wireless communication module 130 and/or for causing the wireless communication module 130 to initiate establishing a connection with the user device 104. Additionally or alternatively, before switching into the low-power mode, the controller 120 may send to the wireless communication module 130 at least some data from the data entry indicative of the injection and/or state of the drug delivery device 102 via, for example, the bus 128 and/or the processor interface 210. Thus, in some implementations, after completing the injection in the controller-on state 320, the drug delivery device 102 may transition into the both-on state 330 before the controller 120 switches into the low-power mode.

The wireless communication module 130 may operate in the active mode during the injection or may switch into the active mode upon the completion of the injection. In some implementations, the wireless communication module 130 switches into the active mode when it is powered on, reset, and/or initialized. In other implementations, the wireless communication module 130 is configured to power on, reset, and/or initialize into the low-power mode, such as, for example, a sleep mode. The wireless communication module 130 may switch into the active mode in response to a trigger signal generated by the controller 120 after the injection is completed. Thus, if the injection mechanism initiates and/or completes an injection in the controller-on state 320 of the drug delivery device 102, for a short period of time after the completion of the injection, the drug delivery device 102 may operate in the both-on state 330. The short period of operation in the both-on state 330 may be 1 ms, 10 ms, 100 ms, 1s or any other suitable time period and may be sufficient for the wireless communication module 130 to obtain the data sent by the controller 120 operating in the active mode.

In some implementations, the controller 120 switches into the low-power mode after the completion of the injection and before the wireless communication module 130 switches into the active mode or detects an activation trigger. Thus, the drug delivery device 102 may transition into the both-off state 310 from the controller-on state 320 after the completion of the injection. A user action may then cause the activation of the wireless communication module 130 and the transition of the drug delivery device 102 into the wireless-on state 340. For example, in implementations in which the door 112 opens upon the completion of the injection, the user may close the door to activate the wireless communication module 130. More precisely, auxiliary circuitry 126 in cooperation with the door sensor 160a may detect that the door 112 is closed and generate the wireless activation trigger. In some implementations, the auxiliary circuitry 126 generates the wireless activation trigger upon the closing of the door 112 only if the reservoir sensor 160c detects an absence of a cartridge and/or an absence of injectable material in the reservoir 150. Thus, in some implementations, the drug delivery device 102 completes an injection in the controller-on state 320, opens the door 112, transitions into the both-off state 310, and transitions into the wireless-on state once the user removes a used cartridge and closes the door 112.

Upon activation, the wireless communication module 130 may access an injection log and/or the drug delivery device state data records stored in the memory 122 or from the removable storage device 124. From the injection log and/or drug delivery device state data records, the wireless communication module 130 may obtain at least some data from a data entry indicative of a completed injection and/or state of the drug delivery device. In some implementations, the wireless communication module 130 obtains the data from the controller 120, e.g., via the bus 128 and/or the processor interface 210. To obtain the data from the controller 120, the wireless communication module 130 may first send a controller activation trigger to wake-up or activate the controller 120, causing the drug delivery device 102 to transition into the both-on state 330 from the wireless-on state 340. After the wireless communication module 130 obtains at least some data from the data entry indicative of the completed injection, the wireless communication module 130 may initiate an attempt to establish a wireless connection with the user device 104. In some implementations, the wireless communication module 130 may establish a wireless connection with the user device 104 before obtaining the data for transferring to the user device 104, and then obtain the data in response to establishing the wireless connection. Establishing the wireless connection with the user device 104 may proceed over a time interval lasting from less than a second to multiple hours or days. That is, if the paired user device 104 is in range of communication and in a state that allows establishing communication (e.g., running a suitable application), the entire process, including establishing communication and transferring information from the drug delivery device 102 to the user device may be completed, for example, within 0.1-10 seconds. If, on the other hand, the paired user device 104 is not available for establishing communication, the wireless communication module 130 may repeatedly attempt to communicate. The sequence of communication attempts may include bursts of transmissions from the wireless communication module 130, each burst followed by a period of radio silence from the wireless communication module 130. Each burst of transmissions may include intervals during which the wireless communication module 130 may “listen” for a response from the user device 104. In other words, a communication attempt may include multiple transmissions interspersed with listening periods during which the wireless communication module 130 may receive a response from the user device 104. The wireless communication module 130 may make the communication attempts while operating in an “advertising mode,” and the associated bursts of transmissions may be referred to as “advertising.”

During the listening periods of the communication attempts, the wireless communication module 130 may consume more power than during the radio silence periods between the communication attempts. While listening, the wireless communication module 130 may draw power for operating a radio receiver and/or an optical (e.g., infrared) receiver within the wireless communication module 130. Between the communication attempts, however, the receiver may be turned off. Thus, the wireless communication module 130 may have more than two active modes with different power consumption levels. For the purpose of the discussion herein, the low-power mode of the wireless communication module 130 may refer to the mode in which the wireless communication module 130 requires an external (generated outside of the wireless communication module 130) trigger signal to “wake-up” before the processor of the wireless communication module 130 can cause the wireless communication module 130 to transmit or receive a wireless communication.

While the wireless communication module 130 is operating in the active mode and advertising in an attempt to establish a wireless connection with the user device 104, the controller 120 may, independently, switch into an active mode from a low-power mode. For example, the auxiliary circuitry 126 may generate a controller activation trigger based on a user action (e.g., pressing a button, replacing a cartridge, and/or closing the door 112) detected using the sensors 160a-d. The controller 120 may switch into the active mode, activate the drive 140, detect a new completed injection, generate a new data entry, and/or switch back into the low-power mode while the wireless communication module 130 is advertising. Thus, in an exemplary sequence of power state transitions, the drug delivery device 102 may transition: i) from the both-off state 310 into the controller-on state 320 for a first injection, ii) into the both-on state 330 or the both-off state 310 upon completing the first injection, iii) into the wireless-on state 340 to establish a wireless connection with the user device 104, iv) into the both-on state 330 to prepare for a second injection while continuing to establish the wireless connection, v) back into the wireless-on state 330 upon completing the second injection, and then vi) into the both-off state upon successfully establishing a connection with the user device 104 and transmitting a message indicative of the first injection and/or the second injection. In some implementations, upon establishing the connection with the user device 104, the wireless communication module 130 sends a controller activation trigger to cause the controller 120 to switch into the active mode. For example, in implementations in which the memory 122 in integrated into the controller 120, the wireless communication module 130 may wake up the controller 120 to request and obtain data stored in the memory 122. In some implementations, the controller 120 and the wireless communication module 130 may complete their corresponding tasks independently and out of sequence, i.e., asynchronously.

Table 1 below shows an exemplary sequence of user actions, corresponding tasks performed by the controller 120 and/or the wireless communication module 130, and the power state of the drug delivery device 102 in which the tasks are performed.

TABLE 1
Power Modes Associated With Various User Actions

| Step | User Action | Controller Task or Mode | WCM Task or Mode | Power State |
|---|---|---|---|---|
| A | None | Low-power mode | Low-power mode | Both-off 310 |
| B | Press a button (e.g. eject) | Wake up | Low-power mode | Both-off 310 |
| | | Complete self-test | | to |
| | | If no errors, open door | | Controller-on 320 |
| C | Insert cartridge and close door | Detect a valid cartridge and | Low-power mode | Controller-on 320 |
| | | cap | | |
| | | Activate indicators | | |
| D | Remove cartridge cap | Detect that cap is removed | Low-power mode | Controller-on 320 |
| | | Turns on target light (not | | |
| | | shown) | | Controller-on 320 |
| E | Touch drug delivery device to | Detect stable injection site | Low-power mode | |
| | injection site and maintains | contact | | |
| | skin contact | Update indicators | | |
| F | Press button while holding | Activate injection mechanism | Low-power mode | Controller-on 320 |
| | drug delivery device to | Update indicators | | |
| | injection site | | | |
| G | Lift drug delivery device off | Generate data entry | Low-power mode | Controller-on 320 |
| | injection site | Open door | | to |
| | | Switch into low-power mode | | Both-off 310 |
| H | Remove cartridge, close door | Low-power mode | Wake up | Wireless-on 330 |
| | | | Initiate advertising | |
| I | Bring a paired mobile (user) | Low-power mode or | Connect to user device | Wireless-on 330 |
| | device with application close to | Perform controller tasks listed | Obtain data (e.g., indicative | or |
| | drug delivery device or bring | above while WCM waits to | of the state of the drug | Both-on 340 |
| | the drug delivery device close | connect to a paired user | delivery device or injection), | to |
| | to a paired user device | device | Transfer message indicative | Both-off 310 |
| | | | of the obtained data | |
| | | | Switch into low-power mode | |

In step A, before any user actions, the drug delivery device 102 is in the both-off state 310. In step B, a user may push a button (e.g., a dedicated eject button or the finger sensor 160b) to generate a controller activation trigger, causing the transition of the drug delivery device 102 into the controller-on state 320. The controller 120 may be configured to perform a self-test or diagnostic routine upon activation and to generate in memory (e.g., the memory 122 and/or the removable storage device 124) a data entry indicative of a state (e.g., an operational state) of the drug delivery device 102 and/or an injection. This data entry may contain information about, for example, the success status of the injection, the number of successful injections, the timing of the injection, the amount of a drug delivered during the injection, the speed (e.g., user-selected speed) of the injection, the timing of the self-test, codes of any detected errors, the remaining charge in the power source 114, the operating mode of the wireless communication module 130, diagnostic information about the sensors 160a-d and indicators 162a, b, and/or other state or status information about the drug delivery device 102 or the nature of the injection. If the controller 120 detects no errors, it may cause the door 112 to open, enabling the user to insert a cartridge with injectable material. In step C, the user may insert a new cartridge and close the door 112. The controller 120 may detect, for example by using the reservoir sensor 160c and the door sensor 160a, that a valid cartridge is inserted and the door 112 is closed. In response, the controller 120 may activate the audio and/or visual indicators 162a, b to indicate to the user to proceed with the following step. In some implementations and/or scenarios, the user closes the door 112 with no cartridge in the drug delivery device 102 to cause the controller 120 and/or the auxiliary circuit 126 to generate a wireless activation trigger and/or a communication trigger. In response, the wireless communication module 130 may wake-up, initiate advertising, and, when connected with the user device 104, transfer a message to the user device 104.

With the valid cartridge loaded and the door 112 closed, the user may proceed with step D of Table 1 by removing the cap 154 from the cartridge. The controller 120 may detect, using a cap sensor (not shown), that the cap 154 is removed and turn-on a target light (not shown) to illuminate the injection site. In step E, the user may touch the drug delivery device 102 to the injection site and maintain contact. Upon detecting stable injection site contact using the skin sensor 160d, the controller 120 may use the indicators 162a, b to indicate to the user to proceed with the injection. In step F, the user may press a button (e.g., finger sensor 160b or a dedicated start button). The controller 120 may detect that the button is pressed while the contact is maintained and activate the injection mechanism. The injection mechanism may use the drive 140, the plunger 142, and/or other components (not shown) to insert the needle 152 and express the injectable material from the reservoir 150 through the needle 152, delivering the subcutaneous injection. The injection mechanism may then retract the needle 152, and the controller 120 may update the indicators 162a, b to inform the user that it is safe to lift off the drug delivery device 102 from the injection site. In step G, the user may lift the drug delivery device 102 off the skin, and the controller 120 may detect that the drug delivery device 102 is no longer in contact with the skin using the skin sensor 160d. In response to detecting the end of the injection and the loss of contact with the skin, the controller 120 may generate in memory (e.g., the memory 122 and/or the removable storage device 124) a data entry indicative of the injection. The data entry may include the time of the injection, the success indication of the injection, information about the cartridge or the injectable material, etc. The controller 120 may cause the door 112 to open prior to or after generating the data entry, and switch into the low-power mode, causing the drug delivery device 102 to transition into the both-off state 310.

In step H, the user may remove the used cartridge and close the door 112, to cause the auxiliary circuit 126 to generate a wireless activation trigger and/or a communication trigger. In response, the wireless communication module 130 may wake-up, causing the drug delivery device 102 to transition into the wireless-on state 330. Once in the active mode, the wireless communication module 130 may initiate advertising. In step 1, the user may bring the drug delivery device 102 sufficiently close to the paired user device 104 or bring the user device 104 sufficiently close to the drug delivery device 102. With the paired user device 104 in range, the wireless communication module 130 may establish a wireless connection with the user device 104. In some implementations, the wireless communication module 130 obtains at least some of the data from the data entry generated by the controller 120 while the controller 120 is in the low-power mode by accessing the memory location of the data entry. In other implementations, the wireless communication module wakes up the controller 120 to help obtain the data, causing the drug delivery device 102 to transition into the both-on state 340. The wireless communication module 130 may obtain the data prior to or after establishing the wireless connection with the user device 104. Because a sequence of attempts to establish the connection with the user device 104 may take the length of the communication time window (which can be several hours or days), the drug delivery device 102 remains available for injections. More specifically, the controller 120 may wake up in response to a user action, as in step B, control one or more additional injections and/or generate one or more additional data entries while the wireless communication module 130 is advertising. After obtaining at least some of the data from the one or more data entries and establishing the wireless connection, the wireless communication module 130 may transfer a message indicative of the obtained data to the user device 104. Upon transferring the data, the wireless communication module 130 may switch into the low-power mode, causing the drug delivery device 102 to transition into the both-off state 310. As described above, analogous user actions (e.g., pressing a button or a finger sensor 160b, closing the door 112, etc.) may cause or trigger different tasks, depending on a scenario. For example, closing the door 112 with the cartridge in the drug delivery device 102 may be a step in preparing for an injection. On the other hand, closing the door 112 without the cartridge in the drug delivery device 102 may trigger an attempt at transferring data to the user device 104. Thus, analogous user actions may be overloaded with multiple consequences to simplify design and enable convenient operation of the drug delivery device 102.

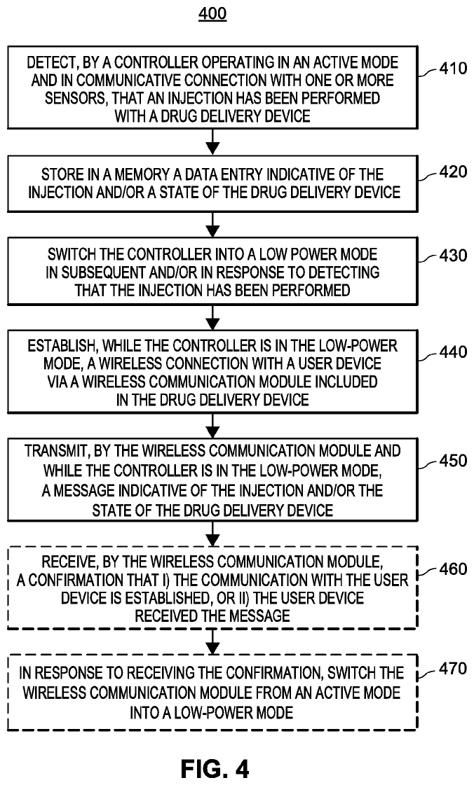

FIG. 4 is a flow diagram of an exemplary method 400 of power-efficient operation of a drug delivery device (e.g., the drug delivery device 102 of FIG. 1). The method 400 may be implemented by processing components of a controller (e.g., the controller 120), a wireless communication module (e.g., the wireless communication module 130), and/or auxiliary circuitry (e.g., auxiliary circuitry 126) in cooperation with sensors (e.g., sensors 160a-d) of the drug delivery device. The controller may be configured, as discussed above, to operate in an active mode or in a low-power mode. Analogously, the wireless communication module may be configured to operate in an active mode or in a low-power mode. Enabling the method 400 of power-efficient operation of the drug delivery device may include configuring the controller and the wireless communication module of the drug delivery device to substantially minimize the time operating in the corresponding active modes. To that end, the controller and the wireless communication module may operate independently and asynchronously. That is, the controller may control components of the drug delivery device to complete injections and generate data entries indicative of the injections or state of the drug delivery device while the wireless communication module is in a low-power mode. The wireless communication module may, on the other hand, establish a wireless connection with a user device (e.g., the user device 104) while the controller is in the low-power mode. Additionally or alternatively, the controller and the wireless communication module may switch from low-power to active modes and/or vice versa while the other is in either mode. Furthermore, the method 400 may include making injections and establishing wireless connections with the user device in response to user actions and/or availability of the user device, and not in a specific sequence.

At block 410, the method 400 may include detecting, by the controller operating in the active mode and in communicative connection with one or more sensors (e.g., sensors 160a-d), that an injection has been performed with the drug delivery device. As discussed above, the controller may control various steps of an injection. After initiating the injection and activating a drive (e.g., drive 140) to express the injectable material from a reservoir (e.g., the reservoir 150), and to deliver the material subcutaneously at an injection site on the skin of a user, the controller may monitor the one or more sensors. In some implementations and/or scenarios, the sensors may indicate that the injection is completed at least in part by detecting that the reservoir is empty and that the drug delivery device lost contact with the skin of the user. In some implementations and/or scenarios, the method 400 may include detecting a failed injection, i.e., that an injection was initiated, but did not complete correctly. For example, the sensors may indicate that the drug delivery device lost contact with the skin of the user before the reservoir was emptied of the injectable material. The method 400 may treat a failed injection as a completed (but unsuccessful) injection and indicate the failure and/or a failure mode in a data entry, generated at block 420 (discussed below).

At block 420, the method 400 may include generating by the controller and storing in memory (e.g., memory 122 and/or removable storage device 124) a data entry indicative of the injection and/or the state of the drug delivery device. The data entry may indicate time of the injection, speed of the injection selected by the user, an amount of drug delivered to the patient during the injection, success status of the injection, state of the drug delivery device at the time of the injection, state of the drug delivery device determined in a self-test routine, and/or other information relating to the injection and/or use of the drug delivery device. In some implementations, the controller starts a data entry upon initiating the injection and completes the entry upon detecting that the injection is completed, successfully or otherwise. The one or more data entries may form an injection log and/or a device diagnostics log.

At block 430, the method 400 may include switching the controller into the low-power mode subsequent and/or in response to detecting that the injection has been performed. The low-power mode of the controller may be a sleep mode, a hibernation mode, a stand-by mode or any other mode that consumes less power than the active mode of the controller by limiting the resources (e.g., reducing access to inputs and/or outputs, powering down memory and/or other peripherals, etc.) of the controller, speed of processing elements of the controller, and/or a set of operations available to the controller. For example, the operation of the controller in the low-power mode may be limited to monitoring a set of inputs on which a wake-up signal (e.g., a controller activation trigger) may be received. In some implementations, the controller is switched-off (e.g., disconnected from the power source 114 using a switch in the power connection 116a) in the low-power mode of the controller. Upon detecting that the injection is completed and before switching into the low-power mode, the controller may perform a set of tasks besides generating and storing the data entry. In some implementations, the method 400 includes the controller sending one or more signals and/or messages to the wireless communication module before switching into the low power mode. The controller may send to the wireless communication module a signal indicative of a wireless activation trigger, a signal indicative of a wireless communication trigger, and/or a message indicative of and/or including at least a portion of the data entry. The controller may send the signals and/or messages via a processor interface (e.g., the processor interface 210 which may be or may include a UART) and/or via activation lines (e.g., the WCM activation line 212 and the controller activation line 214). For example, the controller may send a signal via an activation line to trigger the wireless communication module to switch from the low-power mode to the active mode of the wireless communication module. The controller may send, via the processor interface, a message including or based on at least a portion of the data indicative of the injection and/or a signal to trigger the wireless communication module to initiate one or more communication attempts with a user device (e.g., the user device 104).

The method 400 may include obtaining, by the wireless communication module, at least some data from the data entry generated by the controller and/or stored in the memory location. The wireless communication module may obtain, even while the controller is in the low-power mode, the at least some data from the data entry directly from memory (e.g., the memory 122) and/or a removable storage device (e.g., the removable storage 124) which is in communicative connection with both the wireless communication module and the controller. Additionally or alternatively, the wireless communication module may obtain the portion of the data or information indicative of the data by communicating with the controller via, for example, the processor interface. To enable the wireless communication module to obtain the data, the method 400 may include sending, by the wireless communication module, a signal indicative of a controller activation trigger via the processor interface (e.g., the processor interface 210) or an activation line (e.g., the controller activation line 214). The controller, operating in the low-power mode, may detect the controller activation trigger, and in response switch into the active mode. With the controller operating in the active mode of the controller, the wireless communication module may obtain the portion of the data entry from the controller via the processor interface.

At block 440, the method 400 may include establishing, while the controller is in the low-power mode (e.g., operating in the low-power mode or turned off), a wireless connection with the user device via the wireless communication module included in the drug delivery device. The process of establishing the wireless connection with the user device may continue while the controller is operating in the low-power mode, operating in the active mode, and/or switching between operating modes. The method 400 may include initiating, in response to a communication trigger, a sequence of one or more communication attempts aimed at advertising to a user device that the wireless communication module is ready to establish the wireless connection and/or to transfer information. The advertising transmissions may include information identifying the drug delivery device and/or a user of the drug delivery device. If a user device that is paired with the drug delivery device is in range and ready to communicate (e.g., with the help of the application running on the user device), the user device may send an acknowledgement. The user device may send the acknowledgement in response to recognizing the drug delivery device or the user information in the advertising transmission. The acknowledgement may, in turn, include information identifying the user device and/or the user of the user device. The method 400 may include establishing the connection in response to the wireless communication module permitting the connection based on recognizing the identifying information sent by the user device. If the wireless communication module does not establish a connection after a threshold time period, the wireless communication module may stop the communication attempt and, after a pause, may again start advertising, as discussed in more detail above.

The method 400 may include stopping communication attempts and switching the wireless communication module of the drug delivery device into a low-power mode in response to determining that a communication time window expired. Determining that the communication time window expired may comprise determining that the wireless connection with the user device was not established after at least one of i) a time period greater than a threshold time period, or ii) a number of attempts greater than a threshold number of attempts. Determining that the wireless connection with the user device was not established may, in turn, comprise not receiving any acknowledgements or confirmations from a user device.

Operating in the low-power mode, the wireless communication module may detect a wireless activation trigger and switch into the active mode in response to receiving the wireless activation trigger. A signal indicative of the wireless activation trigger may be sent by the controller via the processor interface and/or an activation line. In some implementations of the method 400, an auxiliary circuit (e.g., the auxiliary circuit 126), in cooperation with sensors, generates the wireless activation trigger. For example, a logic circuit within the auxiliary circuitry may be in communicative connection with the activation line for the wireless communication module. Thus, the wireless activation trigger may be detected in response to a user action, such as pushing a button and/or engaging a finger sensor, opening a compartment (e.g. opening the door 112), and/or closing the compartment of the drug delivery device. In some implementations, the wireless activation trigger is also the communication trigger. The wireless communication module may be configured to start advertising in response to switching into the active mode.

At block 450, the method 400 may include transmitting, by the wireless communication module and while the controller is in the low-power mode, a message indicative of the injection and/or the state of the drug delivery device. The message may include, for example, the success status of the injection, the number of successful injections (if multiple have been performed since the last data transfer), the timing of the injection, the amount of drug delivery during the injection, battery charge remaining, the speed (e.g., user-selected speed) of the injection, the timing of any self-tests, codes of any detected errors, results of any diagnostic or self-test routines, and/or other state or status information about the drug delivery deice or the nature of the injection. In some implementations, an application running on the user device requests certain information from the drug delivery device. For example, the application may keep a log of injections and/or injection attempts in the memory (e.g., memory 186) of the user device and the requested information may allow the application to sync the information stored on the user device with the information stored on the drug delivery device. The synced information may be presented to a user via the display (e.g., the display 188) or may be shared with a healthcare service provider, for example. The user device may also generate one or more informational prompts based on the message received from the drug delivery device.

Some implementations may additionally include block 460 and block 470. At block 460, the method 400 may include receiving, by the wireless communication module, a confirmation that i) the communication with the user device is established, or ii) the user device received the message. At block 470, the method 400 may include, in response to receiving the confirmation, switching the wireless communication module into the low-power mode. In the case of receiving the confirmation that the communication with the user device is established, the wireless communication module may be configured to switch into the low-power mode only after a predetermined period of time, sufficient to transfer information to the user device. In some implementations, the wireless communication module of the drug delivery device and a wireless communication module of user device are Bluetooth and/or Bluetooth low energy (BLE) modules and, accordingly, follow the Bluetooth and/or BLE protocols for establishing a connection. The confirmation of establishing the connection may be a part of the Bluetooth or BLE protocol.

In some implementations, the drug delivery device may perform at least some of the actions described with reference to blocks 440-470 while the controller of the drug delivery device is operating in the active mode and/or before an injection is completed. With respect to the method 400, the drug injection device may perform some of the actions in blocks 440-470 before block 430. Furthermore, the drug delivery device may use the wireless communication module to transmit injection status or drug delivery device state data without retaining (e.g., in the memory of the drug delivery device) the record of the transmissions or the data included in the transmission. In some implementations, the drug delivery device may stream, using the wireless communication module, the injection status or the drug delivery device state data at regular time intervals (e.g., every 1, 10, 100, 1000 ms), at times, while the controller is active. In other implementations, the drug delivery device may send, using the wireless communication module, the injection status or the drug delivery device state data in response to a change detected by one or more of the sensors. The user device, upon receiving the injection status or the drug delivery device state data (from the drug delivery device by way of the wireless communication module), may store the received information and/or generate outputs to a user. Signals generated by the user device may, for example, guide the user in performing the injection. The user may take actions (e.g., pause, continue, or correct user-implemented injection steps) that cause the drug delivery device to change the state, triggering, in some implementations, new communications. In this manner, the wireless communication module may facilitate interactive feedback between the drug delivery device and the user during an injection or during other user-performed actions (e.g., replacing batteries, replacing a cartridge, setting time, etc.).

As discussed above, the controller and the wireless communication module may switch between corresponding active and low-power modes in response to triggers. Some triggers may be based on tasks performed automatically by the controller and/or the wireless communication module. Other triggers may be based on user actions. In some implementations and/or scenarios, the same user actions generate different triggers. Overloading the same user actions with different triggers, depending on scenarios, may enable a simplified design and/or operation of the drug delivery device.

While the foregoing embodiments of the drug delivery device have been described primarily as being an autoinjector or other device that is held in the patient or user's hand over the course of an injection, the scope of the present disclosure is not limited to such hand-held devices. In alternative embodiments, the drug delivery device may be releasably attached to the patient's skin such that the drug delivery device can be worn on the patient's skin during drug delivery, instead of being held in the patients hand. Such a drug delivery device is referred to in some contexts as an on-body injector. On-body injectors can be useful where drug delivery is to occur over tens of seconds, minutes, or hours, and/or in situations where holding the drug delivery device in one's hand over the entire duration of injection is not practical. In such embodiments, an exterior surface of the housing of the drug delivery device may include an adhesive for adhering to the patients skin. Furthermore, in such embodiments, the drug delivery device may have a generally low-profile shape (e.g., a rectangular box) such that the drug delivery device does not impede the patients movement while it is worn by the patient. A low-profile shape may be facilitated by having the longitudinal axis of the delivery member, or at least the pointed end of the delivery member, arranged perpendicular or otherwise non-parallel to the longitudinal axis of the reservoir and/or the longitudinal axis of the housing. Furthermore, the opening in the housing through which the pointed end of the delivery member extends in the delivery state may be covered by a pierceable septum for sterility purposes. In the initial state, the pointed end of the delivery member may not pierce through, or may pierce only partially through, this septum; whereas, in the delivery state, the pointed end of the delivery member may pierce entirely through the septum for insertion into the patient. Furthermore, in an on-body injector configurations of the drug delivery device, the delivery member may be defined by the combination of a hollow or solid trocar and a soft cannula. During operation, the trocar may be deployed to introduce the soft cannula into that patient and then retracted leaving the soft cannula within the patient's body. Except where differences in operation or structure require otherwise, such on-body injector configurations of the injector may incorporate a same or similar power control scheme as that described above.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgrastim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIIa receptor monoclonal antibody); Actemra® (anti-IL6 Receptor mAb); Avastin® (bevacizumab), HuMax-CD4 (zanolimumab); Rituxan® (rituximab, anti-CD2® mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ3 mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a reservoir adapted to contain a drug;
an injection mechanism coupled with the reservoir to deliver the drug;
a power source;
one or more sensors;
a memory;
a controller powered by the power source and having an active mode and a low-power mode, the controller being configured to:
while operating in the active mode, use the one or more sensors to detect that the injection mechanism has performed an injection,
generate in the memory a data entry indicative of a state of the drug delivery device and/or the injection, and
switch into the low-power mode subsequent to or contemporaneous with detecting that the injection mechanism has performed the injection; and
a wireless communication module powered by the power source and configured to:
establish a wireless connection with a user device while the controller is operating in the low-power mode, and
transmit a message indicative of the state of the drug delivery device and/or the injection to the user device.

2. The drug delivery device of claim 1, the wireless communication module being configured to:
detect a first wireless communication module (WCM) trigger, and
wherein establishing the wireless connection comprises initiating a sequence of one or more communication attempts in response to detecting the first WCM trigger.

3. The drug delivery device of claim 2, wherein:
the controller and the wireless communication module are communicatively connected by a processor interface,
the controller is configured to send a signal indicative of the first WCM trigger via the processor interface, and
detecting the first WCM trigger by the wireless communication module comprises receiving the signal indicative of the first WCM trigger via the processor interface.

4. (canceled)

5. The drug delivery device of claim 3, wherein sending the signal indicative of the first WCM trigger by the controller is at least in part in response to detecting that the injection mechanism has performed the injection.

6. The drug delivery device of claim 2, wherein detecting the first WCM trigger by the wireless communication module comprises detecting, using the one or more sensors, while the controller is in the low-power mode, a user action.

7. The drug delivery device of claim 6, comprising:
a compartment configured to contain a cartridge and/or a button; and
wherein the user action comprises at least one of i) opening a compartment of the drug delivery device, ii) closing the compartment of the drug delivery device, or iii) pressing the button of the drug delivery device.

8. The drug delivery device of claim 1, the wireless communication module being configured to:
receive, while operating in an active mode of the wireless communication module, a confirmation from the user device, wherein the confirmation comprises at least one of i) an acknowledgement of the established wireless connection or ii) an indication that the user device received the message, and
in response to receiving the confirmation, switch into a low-power mode of the wireless communication module.

9. (canceled)

10. The drug delivery device of claim 8, the wireless communication module being configured to:
determine that a communication time window expired;
in response to determining that the communication time window expired, switch into the low-power mode of the wireless communication module.

11.-12. (canceled)

13. The drug delivery device of claim 8, the wireless communication module being configured to:
detect a second WCM trigger, while operating in the low-power mode of the wireless communication module, and
switch into the active mode of the wireless communication module in response to detecting the second WCM trigger.

14. (canceled)

15. The drug delivery device of claim 13, comprising:
at least one of i) a compartment configured to contain injection fluid, or ii) a button;
wherein detecting the second WCM trigger by the wireless communication module comprises detecting a user action using the one or more sensors; and
wherein the user action comprises at least one of i) opening the compartment ii) closing the compartment, or iii) pressing the button.

16. (canceled)

17. The drug delivery device of claim 1, wherein the controller is configured to:
detect a controller activation trigger, while operating in the low-power mode of the controller, and
switch into the active mode of the controller in response to detecting the controller activation trigger.

18. (canceled)

19. The drug delivery device of claim 1, wherein the controller and the wireless communication module are configured to operate asynchronously.

20. (canceled)

21. A method of operating a drug delivery device, the method comprising:
detecting, by a controller operating in an active mode and communicatively coupled to one or more sensors, that an injection has been performed with the drug delivery device;
storing in a memory a data entry indicative of a state of the drug delivery device and/or the injection;
switching the controller into a low-power mode subsequent to or contemporaneous with detecting that the injection has been performed;
establishing, while the controller is operating in the low-power mode, a wireless connection with a user device via a wireless communication module included in the drug delivery device; and
transmitting, by the wireless communication module and while the controller is operating in the low-power mode, a message indicative of the state of the drug delivery device and/or the injection to the user device.

22. The method of claim 21, wherein establishing the wireless connection comprises:
detecting, by the wireless communication module, a first wireless communication module (WCM) trigger, and
initiating a sequence of one or more communication attempts in response to detecting the first WCM trigger.

23. The method of claim 22, comprising:
sending, by the controller, a signal indicative of the first WCM trigger via a processor interface, and wherein
detecting the first WCM trigger by the wireless communication module comprises receiving the signal indicative of the first WCM trigger via the processor interface.

24. (canceled)

25. The method of claim 23, wherein sending the signal indicative of the first WCM trigger by the controller is at least in part in response to detecting that the injection has been performed.

26. The method of claim 22, wherein detecting the first WCM trigger by the wireless communication module comprises detecting, using the one or more sensors, while the controller is in the low-power mode, a user action.

27. The method of claim 26, wherein the user action comprises at least one of i) opening a compartment of the drug delivery device, ii) closing the compartment of the drug delivery device, or iii) pressing a button or engaging a finger sensor of the drug delivery device.

28. The method of claim 21, comprising:
receiving, by the wireless communication module operating in an active mode of the wireless communication module, a confirmation from the user device, wherein the confirmation comprises at least one of i) an acknowledgement of the established wireless connection or ii) an indication that the user device received the message; and
in response to receiving the confirmation, switching the wireless communication module into a low-power mode of the wireless communication module.

29. (canceled)

30. The method of claim 28, comprising:
determining that a communication time window expired;
in response to determining that the communication time window expired, switching the wireless communication module into the low-power mode of the wireless communication module.

31.-32. (canceled)

33. The method of claim 28, comprising:
detecting, by the wireless communication module operating in the low-power mode of the wireless communication module, a second WCM trigger; and
switching the wireless communication module into the active mode of the wireless communication module in response to detecting the second WCM trigger.

34. (canceled)

35. The method of claim 33, wherein:
detecting the second WCM trigger by the wireless communication module comprises detecting a user action using the one or more sensors, and
the user action comprises at least one of i) opening a compartment of the drug delivery device, ii) closing the compartment of the drug delivery device, or iii) pressing a button or engaging a finger sensor of the drug delivery device.

36. (canceled)

37. The method of claim 21, comprising:
detecting, by the controller operating in the low-power mode of the controller, a controller activation trigger; and
switching the controller into the active mode of the controller in response to detecting the controller activation trigger.

38. (canceled)

39. The method of claim 21, wherein:
the controller and the wireless communication module operate asynchronously.
